(19) United States
(12) Patent Application Publication
(10) Pub. No.: US 2025/0107795 A1
(43) Pub. Date: Apr. 3, 2025

(54) LATERAL SUTURE ANCHOR FIXATION SYSTEMS AND METHODS

(71) Applicant: ACTIVAS Orthopedics, Inc., Coronado, CA (US)

(72) Inventors: Brian Bowman, Carlsbad, CA (US); Sachiye Koide, San Diego, CA (US); Colin Murphy, Carlsbad, CA (US); Benjamin Arnold, San Diego, CA (US); Thomas A. Afzal, Coronado, CA (US)

(21) Appl. No.: 18/905,922

(22) Filed: Oct. 3, 2024

Related U.S. Application Data
(60) Provisional application No. 63/542,158, filed on Oct. 3, 2023.

(51) Int. Cl. A61B 17/04; A61F 2/08

(52) U.S. Cl. CPC A61B 17/0401; A61F 2/0811; A61B 2017/0409; A61B 2017/044; A61B 2017/0456; A61B 2017/0464; A61F 2002/0823

(57) ABSTRACT

Exemplary lateral suture anchor fixation systems include a lateral anchor, a lateral anchor inserter, an auxiliary or second anchor, and an auxiliary or second anchor inserter.

### CROSS-REFERENCES TO RELATED APPLICATIONS

This application claims the benefit of U.S. Provisional Patent Application No. 63/542,158 filed Oct. 3, 2023, the disclosure of which is incorporated herein by reference.

### BACKGROUND

Embodiments of the present invention relate generally to suture anchor fixation systems and methods, and in particular instances, to lateral suture anchor fixation systems and methods.

### BRIEF SUMMARY

Exemplary lateral suture anchor fixation systems and methods are disclosed herein.

Additional and related features of embodiments of the present invention can be found in U.S. Provisional Patent Application No. 63/469,649 filed May 30, 2023, Systems For Placement Of Multiple Joined Suture Anchors, U.S. Provisional Patent Application No. 63/469,652 filed May 30, 2023, Graft Loading And Deployment System For Rotator Cuff Repair And Reconstruction, and U.S. Provisional Patent Application No. 63/528,144 filed Jul. 21, 2023, Graft Deployment And Attachment Systems And Methods. Relatedly, it is understood that one or more aspects of the suture anchor fixation systems and/or methods disclosed herein can be used in combination with any of the systems and/or methods described in U.S. patent application Ser. Nos. 18/678,223 and 18/679,041 each filed May 30, 2024, and Ser. No. 18/780,206, 18/780,209, and 18/780,214 each filed Jul. 22, 2024. The content of each of the above listed applications is incorporated herein by reference.

According to some embodiments, a lateral suture anchor fixation system may include a lateral anchor, a lateral anchor inserter, an auxiliary anchor, and an auxiliary anchor inserter. The lateral anchor may include an external fixation feature, an inserter connection feature, an internal fixation feature that mates with the auxiliary anchor, and a cannula. The lateral anchor inserter may include a distal repair suture passing feature and a lateral anchor connection feature. The auxiliary anchor may include an external fixation feature that mates with the lateral anchor, an inserter mating feature, and a cannula. The auxiliary anchor inserter may include a distal auxiliary suture passing feature and an auxiliary anchor connection feature.

A cuff can be first repaired using the rotator cuff repair anchors. On the lateral side of the repair, the repair suture can be passed through the repair suture passing feature of the lateral anchor inserter and fixed by the external fixation feature of the lateral anchor. A second process, which may include graft deployment, can be conducted after the repair is complete. An auxiliary lateral suture or set of sutures, which may include a graft suture or sutures, can be fixed to the lateral anchor by passing the auxiliary suture or sutures through the auxiliary suture passing feature of the auxiliary anchor inserter and mating the external fixation feature of the auxiliary anchor to the internal fixation feature of the lateral anchor.

In another aspect, embodiments of the present invention encompass a suture anchor having an exterior fixation mechanism and an internal cannula. The exterior mechanism can be configured to fixate into bone while also fixating a first suture in place. The cannula can be configured to receive a second anchor which can fixate a second suture between the suture anchor and the second anchor. In some cases, the suture anchor includes polyetheretherketone. In some cases, the second anchor includes an unthreaded section configured for insertion into the suture anchor to orient the second anchor properly for fixation.

In another aspect, embodiments of the present invention encompass systems and methods for performing a surgical procedure, such as a rotator cuff repair procedure. In some cases, a surgical method can include performing a rotator cuff repair using a suture anchor, and fixating a suture from a graft to the suture anchor using a second anchor. In some cases, the suture anchor includes an exterior mechanism for fixating into bone and a cannula into which the second anchor can be placed. In some cases, the suture anchor includes polyetheretherketone. In some cases, the second anchor includes an unthreaded section configured for insertion into the suture anchor to orient the second anchor properly for fixation.

In still another aspect, embodiments of the present invention encompass lateral suture anchor fixation systems and related methods. Exemplary lateral suture anchor fixation systems can include a lateral anchor, a lateral anchor inserter, a second anchor, and a second anchor inserter. In some cases, a lateral anchor includes an exterior mechanism for fixating into bone while also fixating a first suture in place, and a cannula into which the second anchor can be placed which can fixate a second suture between the suture anchor and the second anchor. In some cases, the lateral anchor includes polyetheretherketone. In some cases, the second anchor includes an unthreaded section configured for insertion into the lateral anchor to orient the second anchor properly for fixation. In some cases, the lateral anchor is configured for releasable coupling with the lateral anchor inserter. In some cases, the second anchor is configured for releasable coupling with the second anchor inserter. In some cases, systems can also include a repair suture and a graft suture. In some cases, a lateral anchor inserter can include a repair suture passing mechanism configured to engage with the repair suture. In some cases, the second anchor inserter can include a graft suture passing mechanism configured to engage with the graft suture. In some cases, the lateral anchor includes an internal threaded section, and the second anchor includes an external threaded section that is configured to engage the internal threaded section of the lateral anchor. In some cases, systems can include a repair suture, a graft suture, and a graft. In some cases, the lateral anchor includes an external threaded section configured to engage a tissue of a patient. In some cases, the lateral anchor includes an internal cannula having six sides.

Other objectives of the invention will become apparent to those skilled in the art once the invention has been shown and described. These objectives are not to be construed as limitations of applicant's invention, but are merely aimed to suggest some of the many benefits that may be realized by the apparatus of the present application and with its many embodiments.

### BRIEF DESCRIPTION OF THE DRAWINGS

Inventive features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A depicts aspects of lateral suture anchor fixation systems and methods, according to embodiments of the present invention.

FIG. 1B depicts aspects of lateral suture anchor fixation systems and methods, according to embodiments of the present invention.

FIG. 2A depicts aspects of a lateral anchor and lateral anchor inserter, according to embodiments of the present invention.

FIG. 2B depicts aspects of a lateral anchor and lateral anchor inserter, according to embodiments of the present invention.

FIG. 3A depicts aspects of a lateral anchor with inserter, according to embodiments of the present invention.

FIG. 3B depicts aspects of a lateral anchor with inserter, according to embodiments of the present invention.

FIG. 4A depicts aspects of a lateral anchor, according to embodiments of the present invention.

FIG. 4B depicts aspects of a lateral anchor, according to embodiments of the present invention.

FIG. 5A depicts aspects of a lateral anchor inserter, according to embodiments of the present invention.

FIG. 5B depicts aspects of a lateral anchor inserter, according to embodiments of the present invention.

FIG. 6A depicts aspects of a lateral anchor inserter, according to embodiments of the present invention.

FIG. 6B depicts aspects of a lateral anchor inserter, according to embodiments of the present invention.

FIG. 7A depicts aspects of an auxiliary suture anchor with auxiliary suture inserter, according to embodiments of the present invention.

FIG. 7B depicts aspects of a second suture anchor with second suture inserter, according to embodiments of the present invention.

FIG. 8A depicts aspects of an auxiliary suture anchor with auxiliary suture inserter, according to embodiments of the present invention.

FIG. 8B depicts aspects of a second suture anchor with second suture inserter, according to embodiments of the present invention.

FIG. 9A depicts aspects of an auxiliary screw or anchor, according to embodiments of the present invention.

FIG. 9B depicts aspects of a second screw or anchor, according to embodiments of the present invention.

FIG. 10A depicts aspects of an auxiliary anchor inserter, according to embodiments of the present invention.

FIG. 10B depicts aspects of a second anchor inserter, according to embodiments of the present invention.

FIG. 11A depicts aspects of an auxiliary anchor inserter, according to embodiments of the present invention.

FIG. 11B depicts aspects of a second anchor inserter, according to embodiments of the present invention.

FIG. 12A depicts aspects of a lateral anchor with auxiliary anchor and auxiliary anchor inserter, according to embodiments of the present invention.

FIG. 12B depicts aspects of a lateral anchor with second anchor and second anchor inserter, according to embodiments of the present invention.

FIG. 13A depicts aspects of a lateral anchor with auxiliary anchor, according to embodiments of the present invention.

FIG. 13B depicts aspects of a lateral anchor with second anchor, according to embodiments of the present invention.

FIG. 14 depicts aspects of a lateral suture and lateral anchor inserter, according to embodiments of the present invention.

FIG. 15 depicts aspects of an auxiliary suture and second anchor inserter, according to embodiments of the present invention.

FIG. 16 depicts aspects of a lateral anchor and second anchor, according to embodiments of the present invention.

FIG. 17 depicts aspects of a lateral anchor and graft suture, according to embodiments of the present invention.

FIG. 18 depicts aspects of a lateral anchor and graft suture, according to embodiments of the present invention.

It is to be noted that the appended figures illustrate only exemplary embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION

Specific embodiments of the disclosed devices, systems and methods of use will now be described with reference to the drawings. Nothing in this detailed description is intended to imply that any particular component, feature, or step is essential to embodiments of the invention.

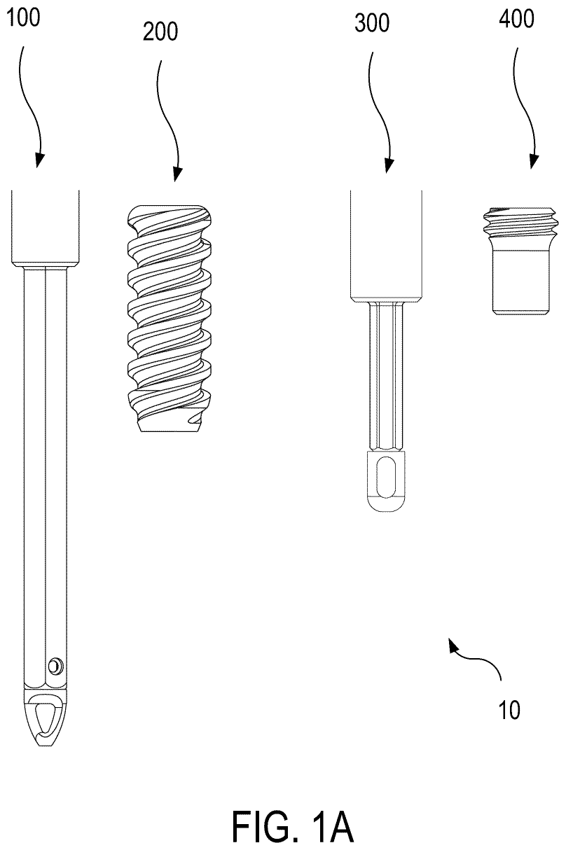

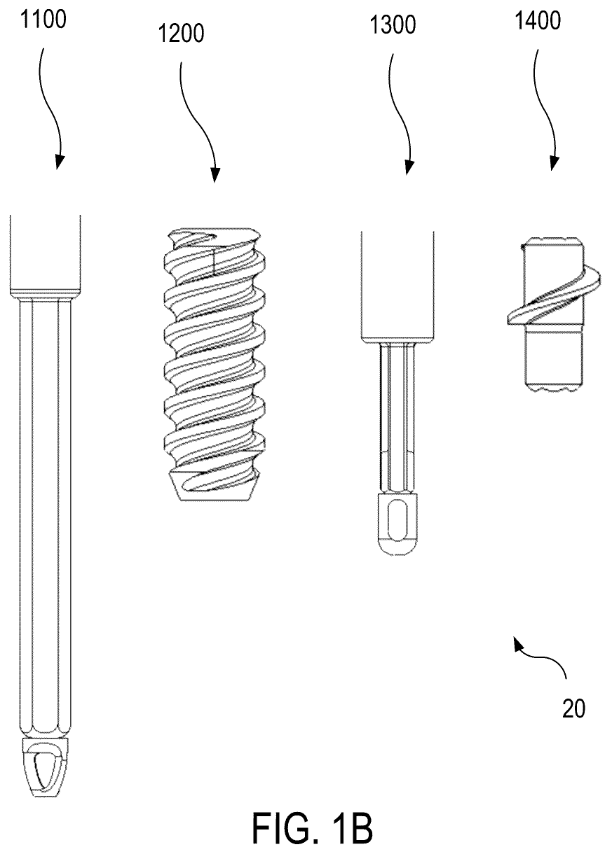

Turning now to the drawings, FIG. 1A depicts aspects of a lateral anchor inserter 100, a lateral anchor 200, an auxiliary anchor inserter 300, and an auxiliary anchor 400, according to embodiments of the present invention. A lateral suture anchor fixation system 10 can include a lateral anchor 200, a lateral anchor inserter 100, an auxiliary anchor 400, and auxiliary anchor inserter 300. In some embodiments, a lateral anchor can be referred to as a suture anchor. In some cases, an auxiliary anchor can be referred to as a second anchor. In some cases, an anchor can include or be made of a thermoplastic material. In some cases, an anchor can include or be made of polyetheretherketone (PEEK).

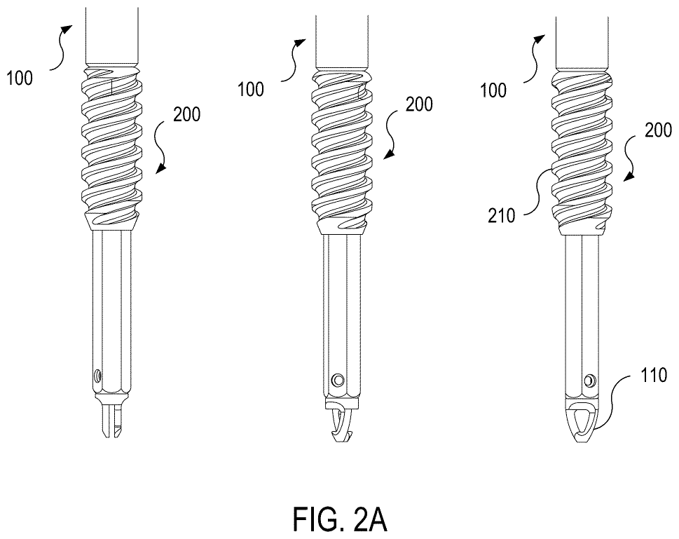

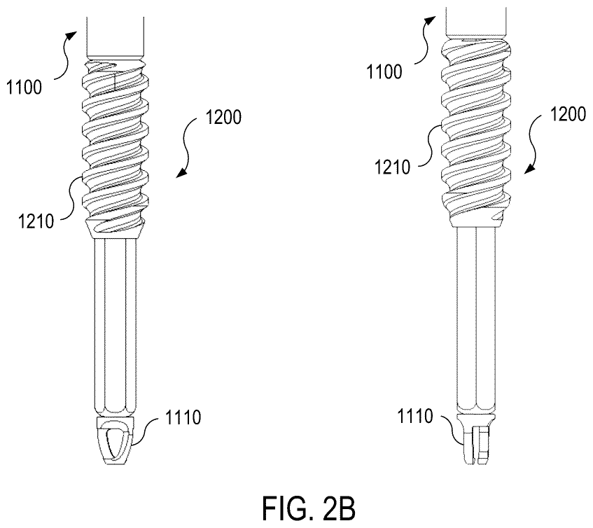

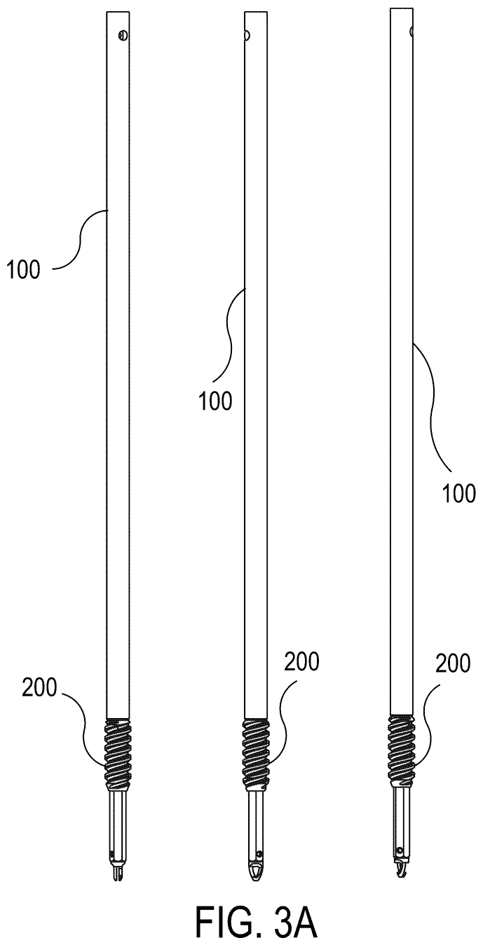

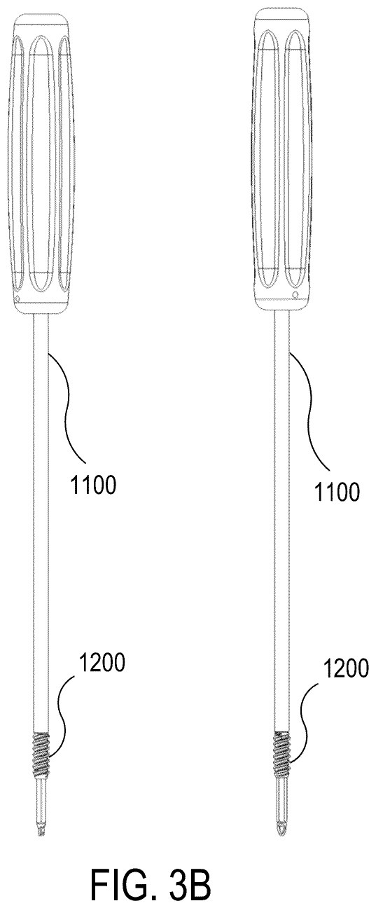

As shown in FIGS. 2A and 3A, a lateral anchor 200 can be loaded or positioned on a lateral anchor inserter 100.

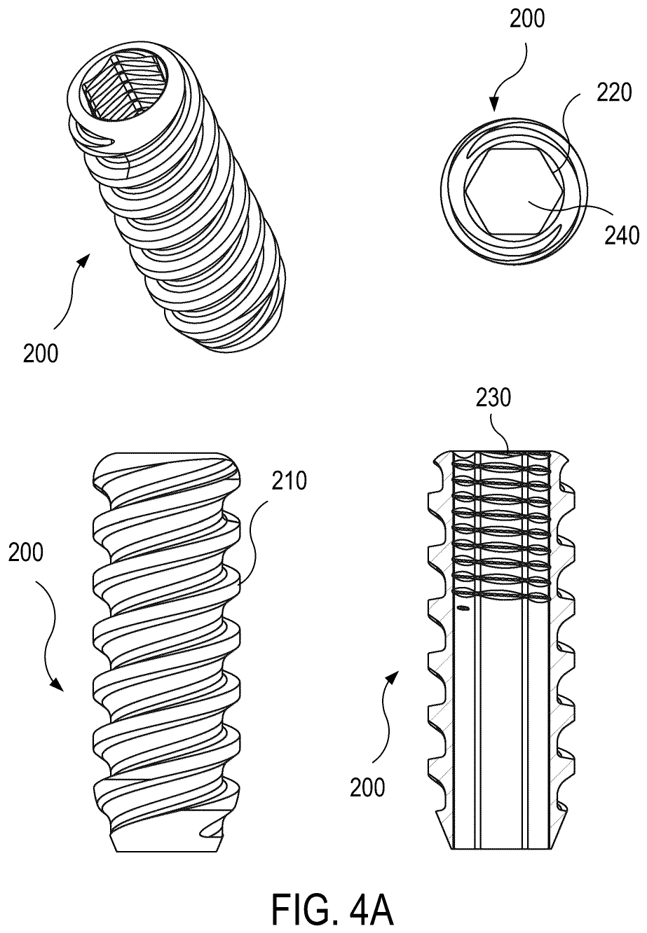

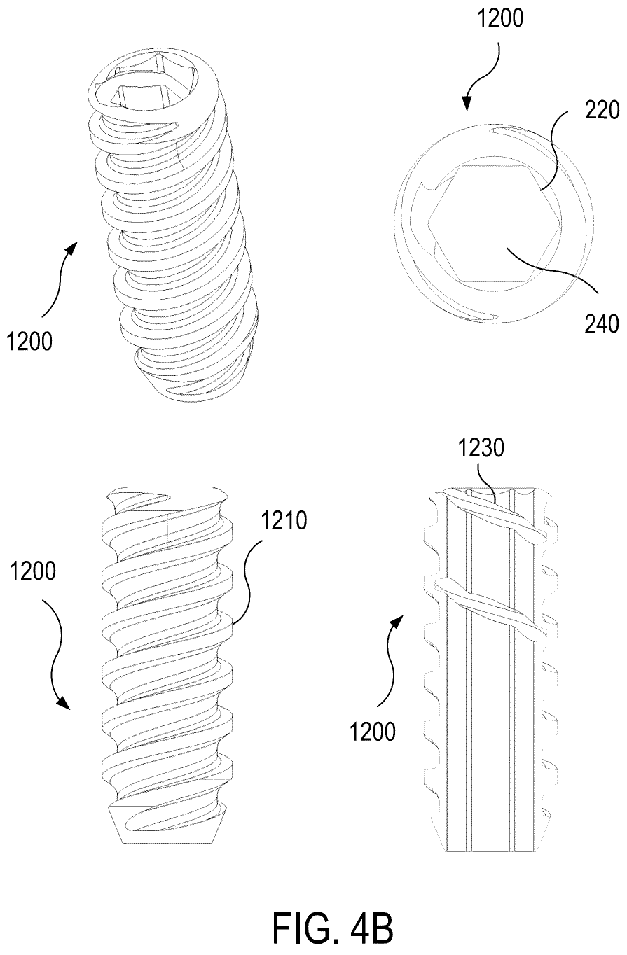

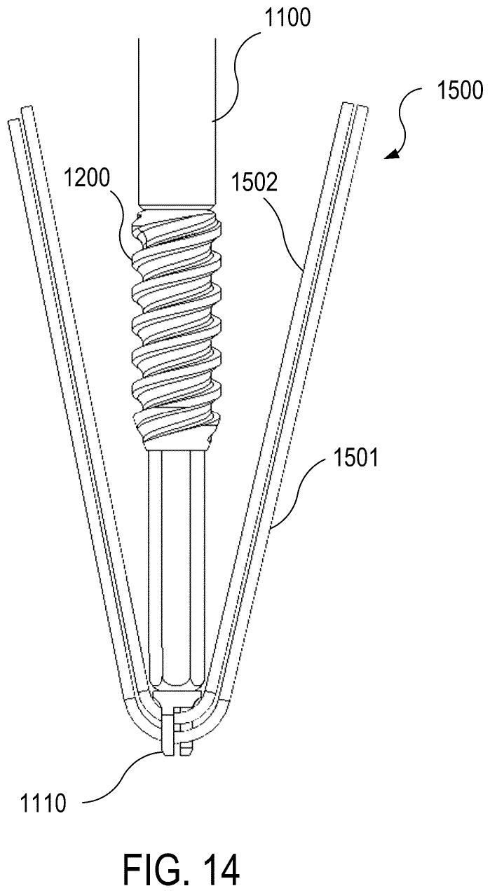

As shown in FIG. 4A, a lateral anchor 200 may be comprised of an external fixation feature or mechanism 210, an inserter connection feature or mechanism 220, an internal fixation feature or mechanism 230 (e.g. internal thread) that mates with an auxiliary anchor, and a cannula or cavity 240. In some embodiments, external fixation mechanism 210 can operate to fixate into bone while also fixating a first suture in place. In some embodiments, for example as depicted in FIGS. 14 to 18 and discussed elsewhere herein, the cannula can operate to receive an auxiliary or second anchor at least partially therein, the auxiliary or second anchor operating to fixate a second suture between the lateral anchor and the auxiliary or second anchor.

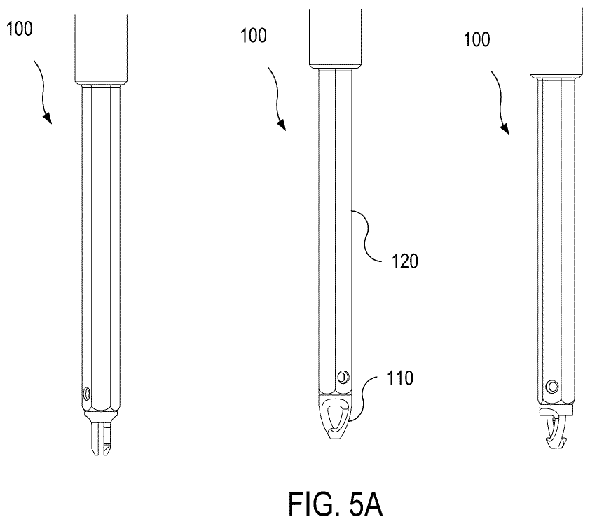

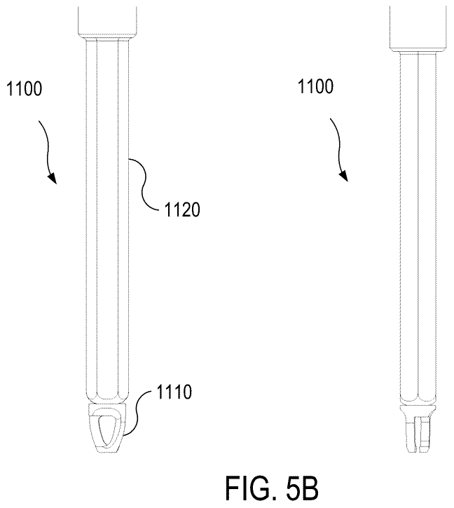

As shown in FIG. 5A, a lateral anchor inserter 100 may be comprised of a distal repair suture passing feature or mechanism 110 and a lateral anchor connection feature or mechanism 120. According to some embodiments, a repair suture can be attached with a lateral or primary anchor and can be oriented with the distal repair suture passing feature or mechanism 110.

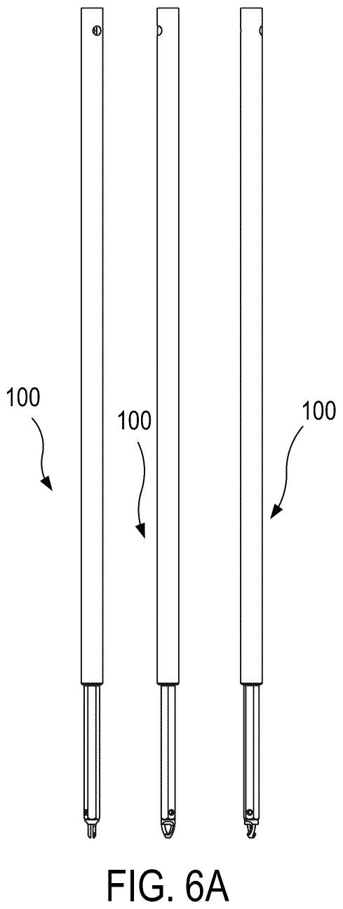

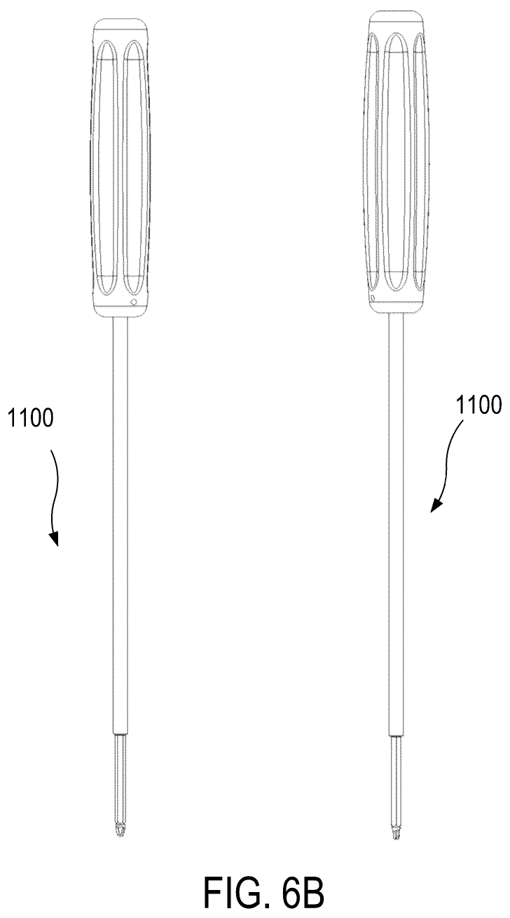

FIG. 6A depicts aspects of a lateral anchor inserter 100.

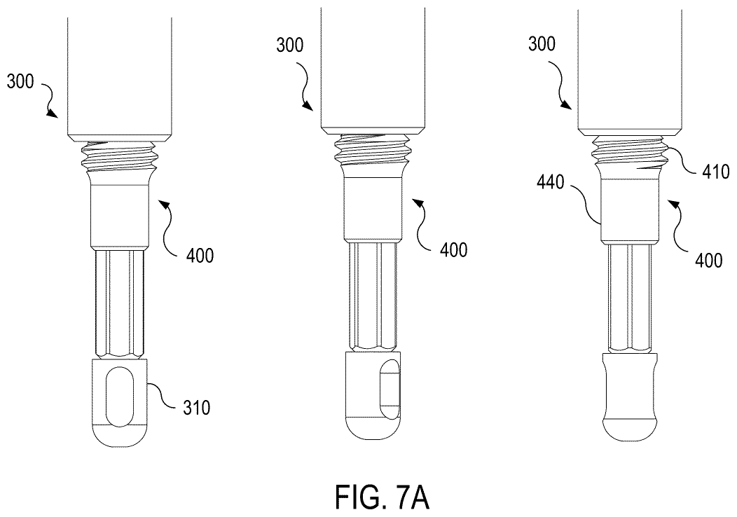

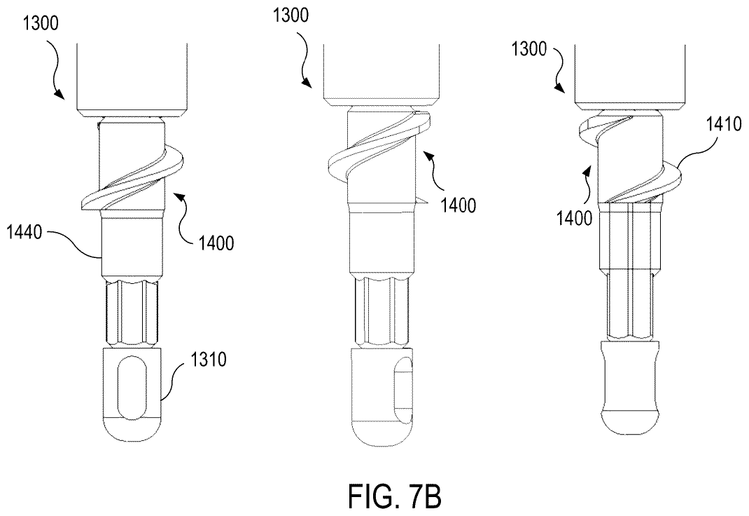

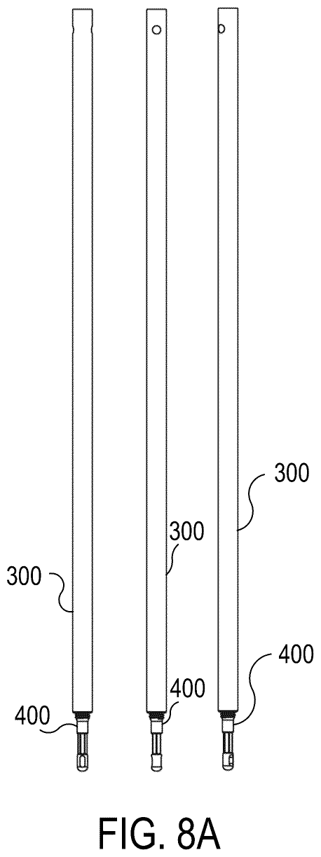

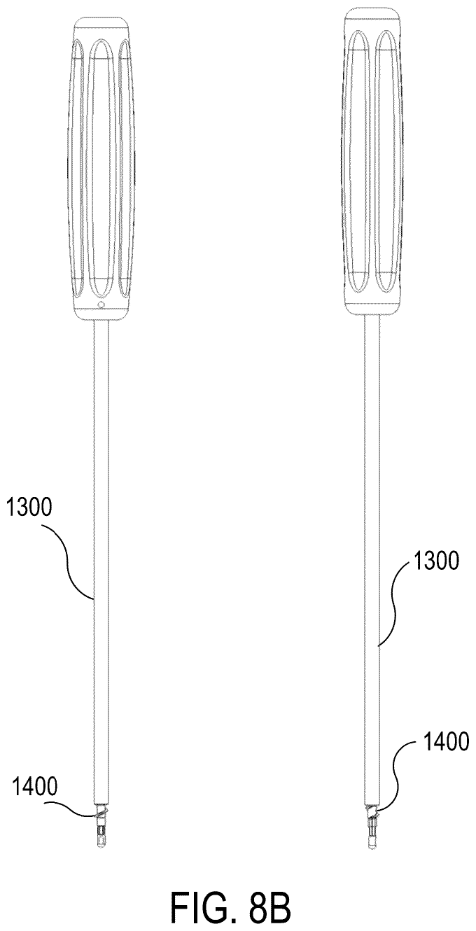

As shown in FIGS. 7A and 8A, an auxiliary suture anchor 400 can be loaded or positioned on an auxiliary anchor inserter 300. In some embodiments, an auxiliary suture anchor 400 can include an unthreaded section 440 configured for insertion into a lateral or suture anchor to orient the auxiliary anchor for fixation.

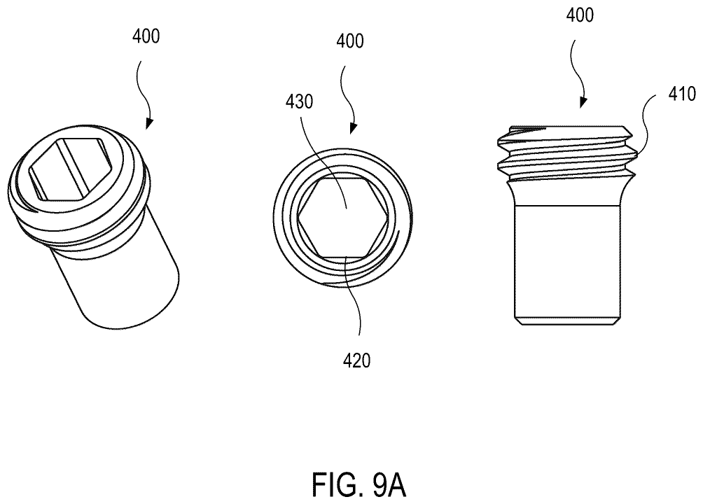

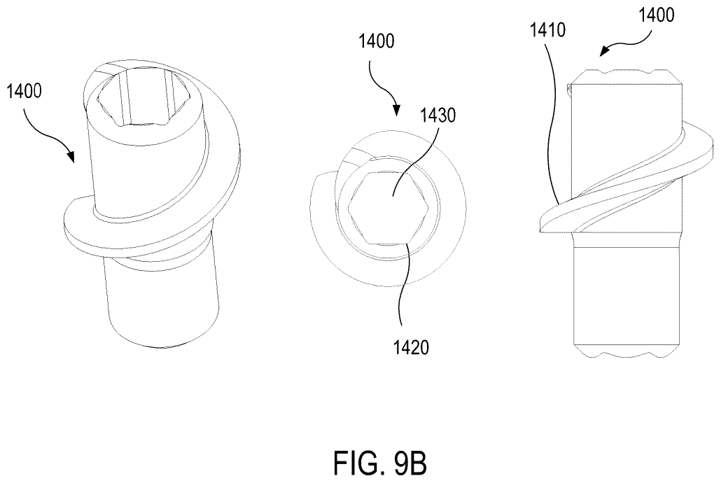

As shown in FIG. 9A, an auxiliary anchor 400 may be comprised of an external fixation feature or mechanism 410 that mates with a lateral anchor, an inserter mating or connection feature or mechanism 420, and a cannula 430. According to some embodiments, an external fixation feature or mechanism 410 can be provided as or include a threaded section. In some cases, an auxiliary anchor 400 can be advanced or screwed in so as to lock a graft suture, for example as further discussed below in reference to FIGS. 7A and 4A.

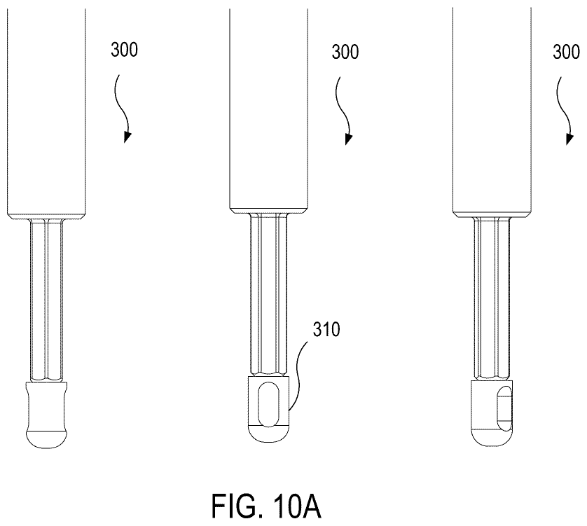

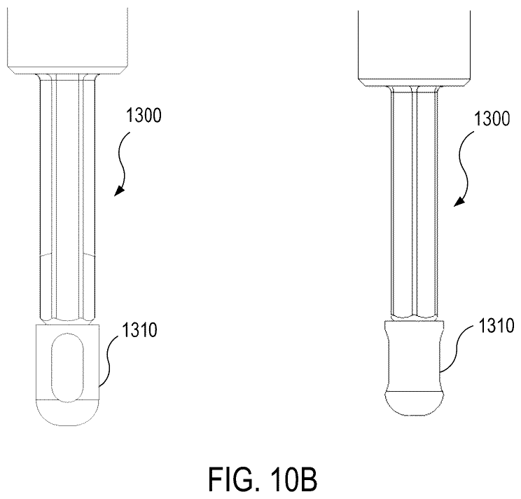

As shown in FIG. 10A, an auxiliary anchor inserter 300 may be comprised of a distal auxiliary suture passing feature or mechanism 310 and an auxiliary anchor connection feature or mechanism 320.

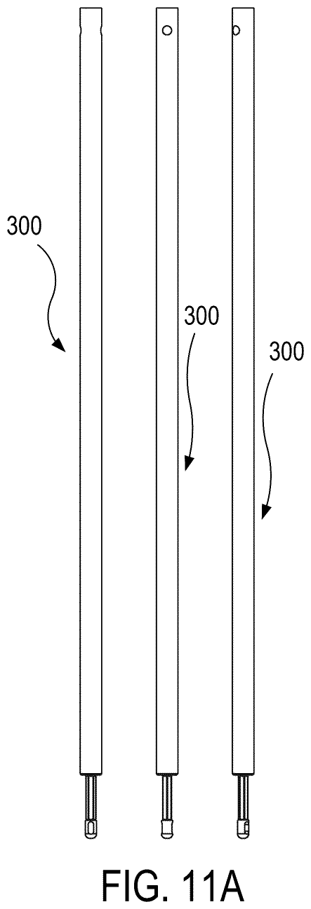

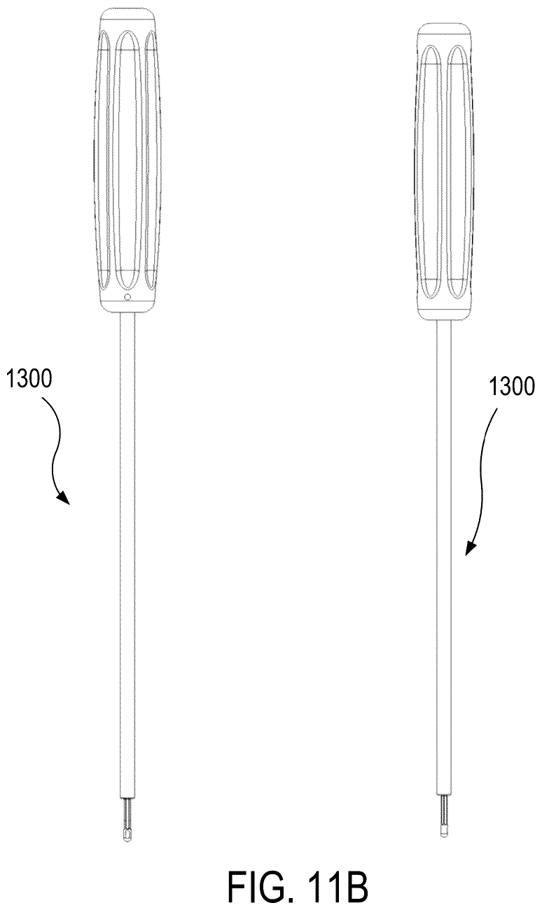

FIG. 11A depicts aspects of an auxiliary anchor inserter 300.

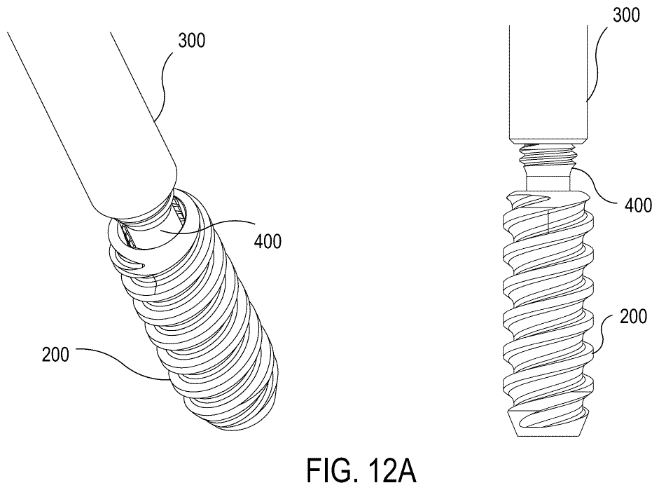

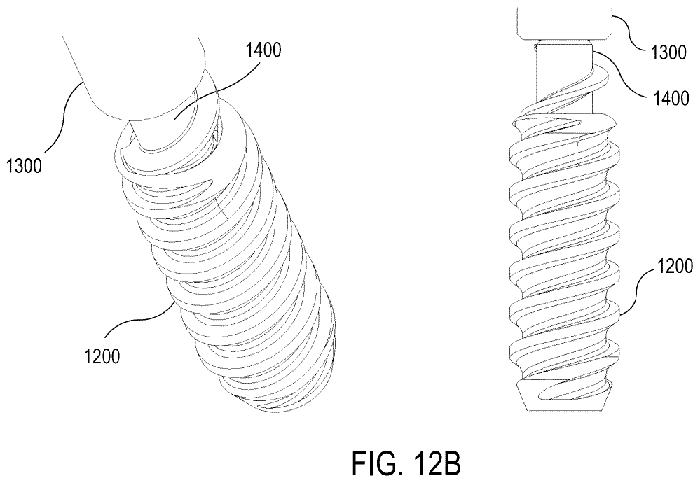

As depicted in FIG. 12A, an auxiliary anchor 400 can be placed on an auxiliary anchor inserter 300, and a lateral anchor 200 can be engaged with the auxiliary anchor 400.

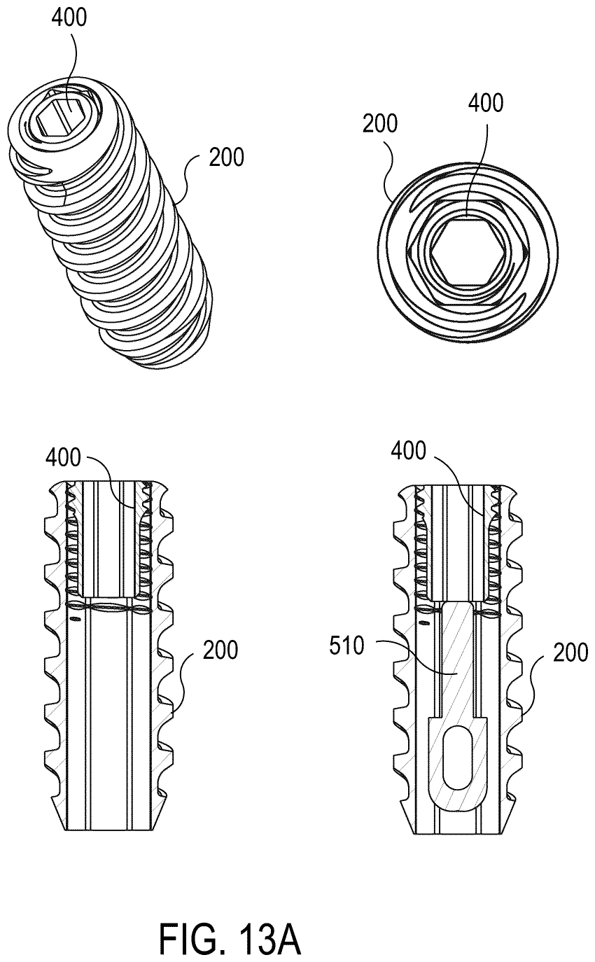

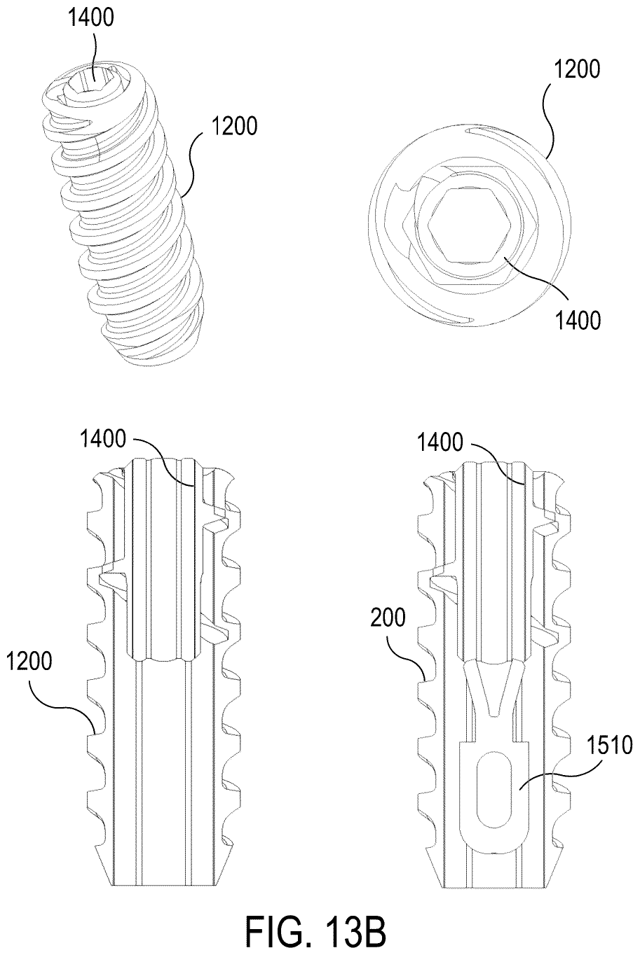

As depicted in FIG. 13A, a lateral anchor 200 can be engaged with an auxiliary anchor 400. A shown here, an auxiliary suture loop 510 can be retained in the lateral anchor 200.

According to some embodiments, a cuff can first be repaired using rotator cuff repair anchors. In some embodiments, a lateral or primary anchor can act as a rotator cuff repair anchors. On the lateral side of the repair, a repair suture can be passed through the repair suture passing feature or mechanism of the lateral anchor inserter and fixed by the external fixation feature or mechanism of the lateral anchor. For example, with reference to FIG. 2A, it can be seen that a repair suture can be passed through the repair suture passing mechanism 110 of the lateral anchor inserter 100, and fixed by the external fixation mechanism 210 of the lateral anchor 200. In some embodiments, for example as depicted in FIGS. 14 to 18 and discussed elsewhere herein, a repair suture can be engaged with a lateral anchor inserter and a lateral anchor.

A second process, which may include graft deployment, can be conducted after the repair is complete. An auxiliary lateral suture or set of sutures, which may be comprised of a graft suture or sutures, can be fixed to the lateral anchor by passing the auxiliary suture or sutures through the auxiliary suture passing feature or mechanism of the auxiliary anchor inserter and mating the external fixation feature or mechanism of the auxiliary anchor to the internal fixation feature or mechanism of the lateral anchor. For example, with reference to FIGS. 7A and 4A, it can be seen that an auxiliary lateral suture can be fixed to the lateral anchor 200 by passing the auxiliary suture through the auxiliary suture passing mechanism 310 of the auxiliary anchor inserter 300 and mating the external fixation mechanism 410 (e.g. external thread) of the auxiliary anchor 400 to the internal fixation mechanism 230 (e.g. internal thread) of the lateral anchor 200. In some embodiments, for example as depicted in FIGS. 14 to 18 and discussed elsewhere herein, a graft suture can be engaged with an auxiliary or second anchor inserter and a lateral anchor, and treatment methods can include fixating a suture from a graft to a suture anchor using an auxiliary anchor.

FIG. 1B depicts aspects of a lateral anchor inserter 1100, a lateral anchor 1200, a second anchor inserter 1300, and a second anchor 1400, according to embodiments of the present invention. A lateral suture anchor fixation system 20 can include a lateral anchor 1200, a lateral anchor inserter 1100, a second anchor 1400, and a second anchor inserter 1300. In some embodiments, a lateral anchor can be referred to as a suture anchor. In some cases, an auxiliary anchor can be referred to as a second anchor. In some cases, an anchor can include or be made of polyetheretherketone (PEEK).

As shown in FIGS. 2B and 3B, a lateral anchor 1200 can be loaded or positioned on a lateral anchor inserter 1100.

As shown in FIG. 4B, a lateral anchor 1200 may be comprised of an external fixation feature or mechanism 1210, an inserter connection feature or mechanism 1220 (e.g. hex shaped), an internal fixation feature or mechanism 1230 (e.g. internal thread) that mates with a second anchor, and a cannula or cavity 1240. In some embodiments, external fixation mechanism 1210 can operate to fixate into bone while also fixating a first suture in place. In some embodiments, the cannula 1240 can operate to receive a second anchor at least partially therein, the second anchor operating to fixate a second suture between the lateral anchor and the second anchor. In some embodiments, for example as depicted in FIGS. 14 to 18 and discussed elsewhere herein, the cannula can operate to receive an auxiliary or second anchor at least partially therein, the auxiliary or second anchor operating to fixate a second suture between the lateral anchor and the auxiliary or second anchor.

As shown in FIG. 5B, a lateral anchor inserter 1100 may be comprised of a distal repair suture passing feature or mechanism 1110 and a lateral anchor connection feature or mechanism 1120. According to some embodiments, a repair suture can be attached with a lateral or primary anchor and can be oriented with the distal repair suture passing feature or mechanism 1110.

FIG. 6B depicts aspects of a lateral anchor inserter 1100.

As shown in FIGS. 7B and 8A, a second suture anchor 1400 can be loaded or positioned on a second anchor inserter 1300. In some embodiments, a second suture anchor 1400 can include an unthreaded section 1440 configured for insertion into a lateral or suture anchor to orient the second anchor for fixation.

As shown in FIG. 9B, a second anchor 400 may be comprised of an external fixation feature or mechanism 1410 that mates with a lateral anchor, an inserter mating or connection feature or mechanism 1420 (e.g. hex shaped), and a cannula 1430. According to some embodiments, an external fixation feature or mechanism 1410 can be provided as or include a threaded section. In some cases, a second anchor 1400 can be advanced or screwed in so as to lock a graft suture, for example as further discussed below in reference to FIGS. 7B and 4B.

As shown in FIG. 10B, a second anchor inserter 1300 may be comprised of a distal second suture passing feature or mechanism 1310 and a second anchor connection feature 1320 (e.g. hex shaped).

FIG. 11B depicts aspects of a second anchor inserter 1300.

As depicted in FIG. 12B, a second anchor 1400 can be placed on a second anchor inserter 1300, and a lateral anchor 1200 can be engaged with the second anchor 1400.

As depicted in FIG. 13B, a lateral anchor 200 can be engaged with an auxiliary anchor 400. A shown here, a second suture loop 1510 can be retained in the lateral anchor 200.

According to some embodiments, a cuff can first be repaired using rotator cuff repair anchors. In some embodiments, a lateral or primary anchor can act as a rotator cuff repair anchors. On the lateral side of the repair, a repair suture can be passed through the repair suture passing feature or mechanism of the lateral anchor inserter and fixed by the external fixation feature or mechanism of the lateral anchor. For example, with reference to FIG. 2B it can be seen that a repair suture can be passed through the repair suture passing mechanism 1110 of the lateral anchor inserter 1100, and fixed by the external fixation mechanism 1210 of the lateral anchor 1200. In some embodiments, for example as depicted in FIGS. 14 to 18 and discussed elsewhere herein, a repair suture can be engaged with a lateral anchor inserter and a lateral anchor.

A second process, which may include graft deployment, can be conducted after the repair is complete. A second lateral suture or set of sutures, which may be comprised of a graft suture or sutures, can be fixed to the lateral anchor by passing the second suture or sutures through the second suture passing feature or mechanism of the second anchor inserter and mating the external fixation feature or mechanism of the second anchor to the internal fixation feature or mechanism of the lateral anchor. For example, with reference to FIGS. 7B and 4B, it can be seen that an auxiliary lateral suture can be fixed to the lateral anchor 1200 by passing the auxiliary suture through the second suture passing mechanism 1310 of the second anchor inserter 1300 and mating the external fixation mechanism 1410 (e.g. external thread) of the second anchor 1400 to the internal fixation mechanism 1230 (e.g. internal thread) of the lateral anchor 200. In some embodiments, for example as depicted in FIGS. 14 to 18 and discussed elsewhere herein, a graft suture can be engaged with an auxiliary or second anchor inserter and a lateral anchor, and treatment methods can include fixating a suture from a graft to a suture anchor using an auxiliary anchor.

With reference to FIG. 14, it is understood that a first suture 1500, which may also be referred to as a primary suture, a lateral suture, or a repair suture, can be passed through a repair suture passing mechanism or distal repair suture passing mechanism 1110 of a lateral anchor inserter 1100. The first suture 1500 can include a distal thread 1501 and a proximal thread 1502.

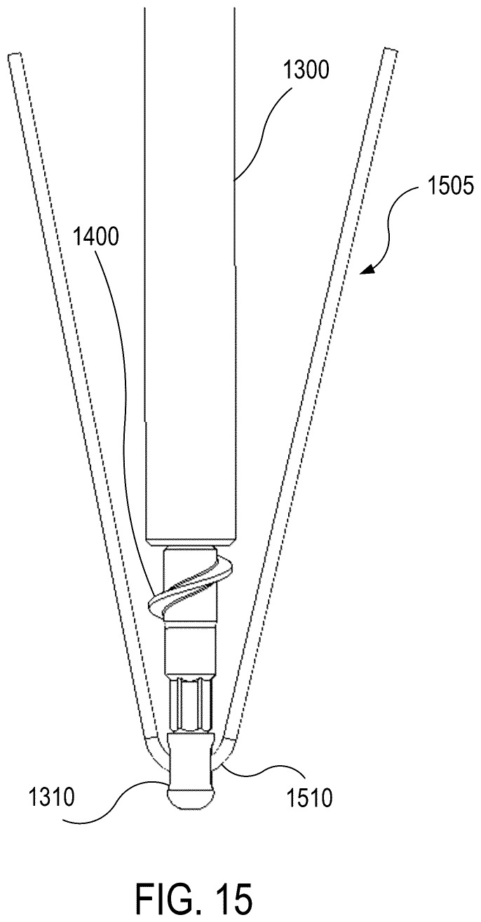

As depicted in FIG. 15, a second suture 1505, which may also be referred to as an auxiliary suture or a graft suture, can be passed through distal second suture passing feature or mechanism 1310 of a second anchor inserter 1300. The second suture 1505 can include a loop 1510.

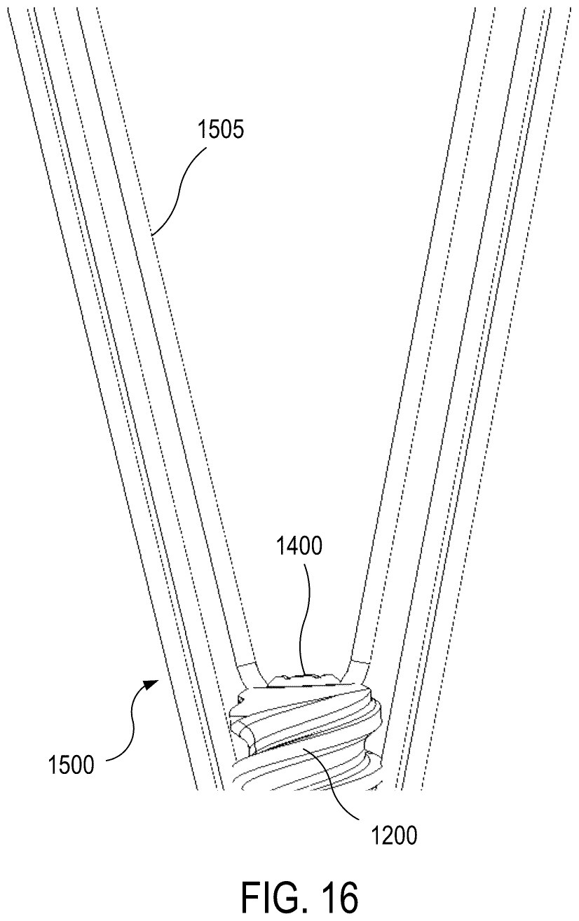

As depicted in FIG. 16, second anchor 1400 can be engaged with a lateral anchor 1200 while a graft suture 1505 is engaged with the second anchor 1400.

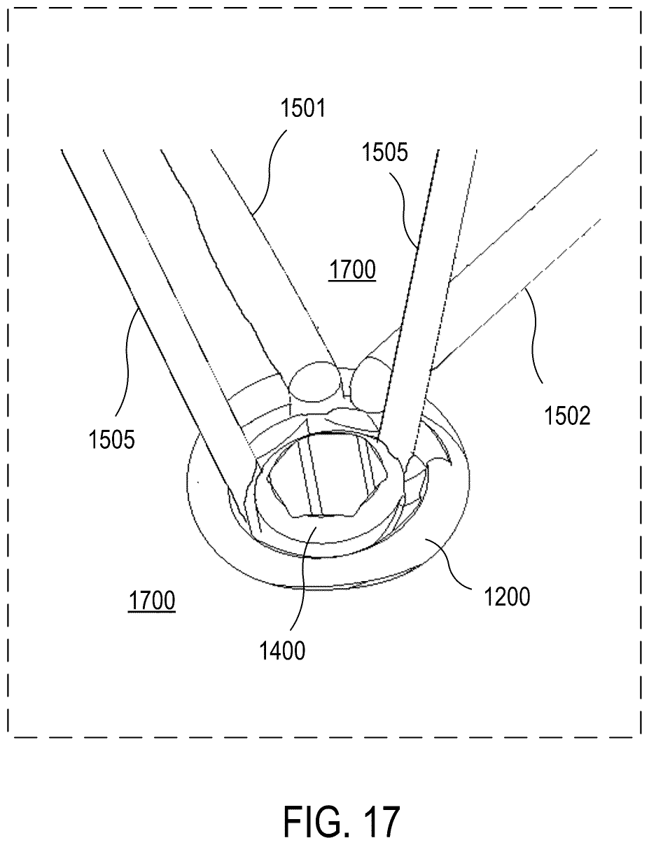

As depicted in FIG. 17, the lateral anchor 1200 can be engaged with a bone 1700 or other tissue of a patient, such that a repair suture (which can include a first or distal thread 1501 and a second or proximal thread 1502) can be positioned or lodged between the lateral anchor 1200 and the bone 1700, and such that a graft suture 1505 can be positioned or lodged between the lateral anchor 1200 and the second anchor 1400 (e.g. on the inside of the lateral anchor and on the outside of the second anchor).

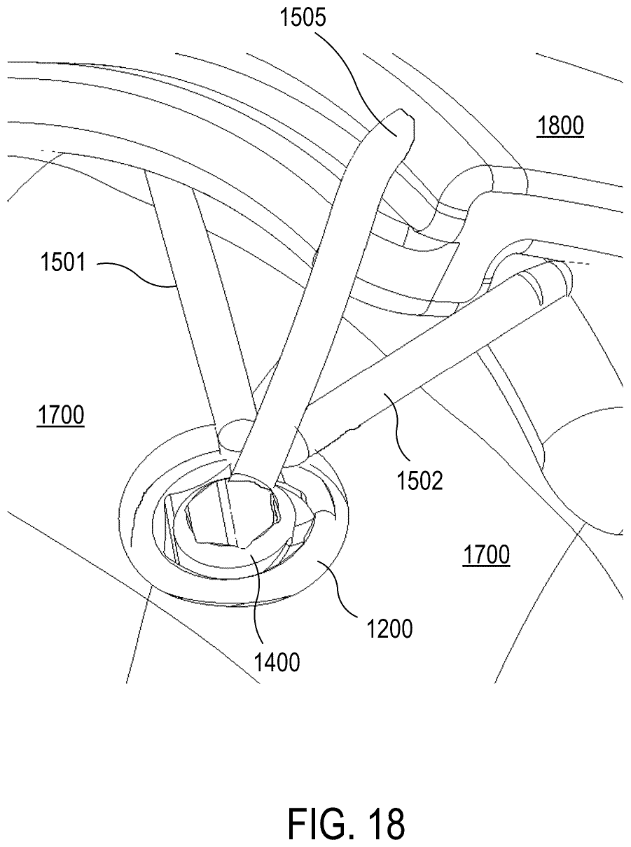

As depicted in FIG. 18, the lateral anchor 1200 can be engaged with a bone 1700 or other tissue of a patient, such that a repair suture (which can include a first or distal thread 1501 and a second or proximal thread 1502) can be positioned or lodged between the lateral anchor 1200 and the bone 1700, and such that a graft suture 1505 can be positioned or lodged between the lateral anchor 1200 and the second anchor 1400 (e.g. on the inside of the lateral anchor and on the outside of the second anchor). The graft suture 1505 can also be coupled or engaged with a graft 1800.

According to some embodiments, a cannula of a lateral anchor 1200 can operate to receive an auxiliary or second anchor 1400 at least partially there, and the auxiliary or second anchor 1400 can operate to fixate a graft suture 1505 between the lateral anchor 1200 and the auxiliary or second anchor 1400. According to some embodiments, a repair suture (which can include first thread 1501 and second thread 1502) can be engaged with the lateral anchor 1200. According to some embodiments, the graft suture 1505 can be engaged with the lateral anchor 1200, and treatment methods can include fixating a suture (e.g. graft suture 1505) from a graft 1800 to a lateral or suture anchor 1200 using an auxiliary or second anchor 1400.

Although the preceding description contains significant detail in relation to certain preferred embodiments, it should not be construed as limiting the scope of the invention but rather as providing illustrations of the preferred embodiments.

Embodiments of the present invention encompass kits having one or more components of a system as disclosed herein. In some embodiments, the kit includes one or more system components, along with instructions for using the component(s) for example according to any of the methods disclosed herein.

All features of the described systems and devices described above and in Appendix A are applicable to the described methods mutatis mutandis, and vice versa.

In addition, each reference provided herein is incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A suture anchor comprising:
an exterior mechanism for fixating into bone while also fixating a first suture in place; and
a cannula into which a second anchor can be placed which can fixate a second suture between the suture anchor and the second anchor.

2. The suture anchor according to claim 1, wherein the suture anchor comprises polyetheretherketone.

3. The suture anchor according to claim 1, wherein the second anchor comprises an unthreaded section configured for insertion into the suture anchor to orient the second anchor properly for fixation.

4. A method of performing a surgical procedure, the method comprising:
performing a rotator cuff repair using a suture anchor; and
fixating a suture from a graft to the suture anchor using a second anchor.

5. The method according to claim 4, wherein the suture anchor comprises an exterior mechanism for fixating into bone and a cannula into which the second anchor can be placed.

6. The method according to claim 4, wherein the suture anchor comprises polyetheretherketone.

7. The method according to claim 4, wherein the second anchor comprises an unthreaded section configured for insertion into the suture anchor to orient the second anchor properly for fixation.

8. A lateral suture anchor fixation system, comprising:
a lateral anchor;
a lateral anchor inserter;
a second anchor; and
a second anchor inserter.

9. The system according to claim 8, wherein the lateral anchor comprises:
an exterior mechanism for fixating into bone while also fixating a first suture in place; and
a cannula into which the second anchor can be placed which can fixate a second suture between the suture anchor and the second anchor.

10. The system according to claim 8, wherein the lateral anchor comprises polyetheretherketone.

11. The system according to claim 8, wherein the second anchor comprises an unthreaded section configured for insertion into the lateral anchor to orient the second anchor properly for fixation.

12. The system according to claim 8, wherein the lateral anchor is configured for releasable coupling with the lateral anchor inserter.

13. The system according to claim 8, wherein the second anchor is configured for releasable coupling with the second anchor inserter.

14. The system according to claim 8, further comprising a repair suture and a graft suture.

15. The system according to claim 14, wherein the lateral anchor inserter comprises a repair suture passing mechanism configured to engage with the repair suture.

16. The system according to claim 14, wherein the second anchor inserter comprises a graft suture passing mechanism configured to engage with the graft suture.

17. The system according to claim 8, wherein the lateral anchor comprises an internal threaded section and the second anchor comprises an external threaded section that is configured to engage the internal threaded section of the lateral anchor.

18. The system according to claim 8, further comprising a repair suture, a graft suture, and a graft.

19. The system according to claim 8, wherein the lateral anchor comprises an external threaded section configured to engage a tissue of a patient.

20. The system according to claim 8, wherein the lateral anchor comprises an internal cannula having six sides.
